(19) United States
(12) Patent Application Publication
(10) Pub. No.: US 2008/0077111 A1
(43) Pub. Date: Mar. 27, 2008

(54) Disposable diaper with unitized standing double cuff structure

(75) Inventors: Edward P. Erdman, West Chester, PA (US); Mariela Muniz, West Chester, PA (US)

Correspondence Address: GOSZ AND PARTNERS LLP, ONE STATE STREET, BOSTON, MA, 02109, US

(73) Assignee: Tyco Healthcare Retail Services, AG

(21) Appl. No.: 11/524,755

(22) Filed: Sep. 21, 2006

(51) Int. Cl. A61F 13/15

(52) U.S. Cl. 604/385.28

(57) ABSTRACT

The present invention provides an improved mechanism for preventing leakage of body exudates from a disposable absorbent article, such as a diaper, by providing a pair of unitized standing double cuff structures placed along laterally opposite sides of absorbent core of the absorbent article. The double cuff structure is made using a single ribbon, with each edge of the ribbon forming a standing leg gather, and the center portion of the ribbon being attached to the top sheet.

### BACKGROUND OF THE INVENTION

The present invention relates generally to disposable absorbent articles. Specifically, the present invention relates to disposable absorbent articles with a unitized standing double cuff structure designed to improve leakage protection.

Disposable absorbent articles such as disposable diapers, training pants, protective underwear, and the like are known. A typical disposable absorbent article frequently makes use of an absorbent core located between a top sheet and a back sheet. The top sheet is commonly formed of a material which is pervious to body fluids, e.g., urine, to allow the transfer of such fluids into the core. The back sheet is commonly formed of a liquid impervious material to form a barrier wall so that any fluid absorbed by the article cannot escape out of the back sheet. The absorbent core is used to absorb the released body exudates by the user wearing the article. Typically, fluff e.g., wood pulp or other cellulosic fiber is used as the material for the absorbent core.

In order to improve the absorbability of the absorbent core, highly absorptive polymers, sometimes referred to as “super-absorbent-polymers” (which may be in the form of particles or fibers), are included with the fluff. However, absorbing rates of the absorbent core are not adequately high enough to absorb a high quantity of liquids at once when such quantity is released onto the upper surfaces of the top sheet and the core. As a result, the liquid, at least partially, flows onto the upper surface of the sheet laterally and often leaks out along the side edges of the article. Such leakage occurs when the quantity of body fluid released is large.

In order to prevent such leakage, leakage-protecting baffles or cuffs are provided on the side edges of articles such as diapers. The cuffs conform the side edges of the diaper to the wearer's body, thereby preventing released body exudates from escaping from the diaper.

U.S. Pat. No. 4,579,556 discloses a disposable diaper in which the opposite side edges of the absorbent core are covered with part of an impervious back sheet, thereby achieving a certain degree of leakage protection. However, there is a possibility of leakage of body exudates when the side edges of the diaper are subjected to pressure by the wearer.

U.S. Pat. No. 4,892,528 discloses a disposable diaper in which two sheets resistant to passage of water are disposed adjacent to and partially outwardly from two side surfaces of absorbent core. The two sheets along with the back sheet are used to make leakage-protecting baffles, which extend above the top surface of the absorbent core. In this invention, there is a risk of releasing body exudates due to leakage once such extended baffles twist due to body movements.

U.S. Pat. No. 5,167,653 discloses a disposable garment provided with a plurality of flaps extending along laterally opposite sides of the garment and normally biased by their own elastic shrinking potential to turn upward. This invention makes the garment complicated and difficult to wear because of multiple flaps containing elastic members.

In light of the above described disadvantages, what is required is a simple to manufacture disposable absorbent article that effectively prevents leakage. Additionally, the disposable absorbent article should prevent liquids from leaking from the article irrespective of body movements or the posture of the person wearing the article.

### SUMMARY OF THE INVENTION

The present invention improves a mechanism for blocking the leakage of body exudates from a diaper by providing a pair of unitized standing double cuff structures placed along laterally opposite sides of the absorbent core of the absorbent article.

In accordance with an embodiment of the present invention, a disposable absorbent article comprises a liquid permeable top sheet, a liquid impermeable back sheet, an absorbent core interposed between the top sheet and the back sheet, and a pair of unitized standing double cuff structures located outwardly with respect to the absorbent core along laterally opposite sides. The unitized standing double cuff structure is made of a single ribbon. A center portion of the ribbon is attached to the topsheet and two edges of the ribbon form standing leg gathers placed on both sides of the center portion.

In one embodiment the height of the standing leg gathers placed on both sides of the center portion of the ribbon are equal.

In an alternate embodiment, the height of the standing leg gather placed nearer to the absorbent core is greater than the height of the standing leg gather placed further away from the absorbent core.

### BRIEF DESCRIPTION OF THE DRAWINGS

These and other features and advantages of the present invention will be appreciated, as they become better understood by reference to the following Detailed Description when considered in connection with the accompanying drawings:

FIG. 1 is a schematic view of a diaper comprising a single cuff design for preventing leakage of body exudates from the diaper;

FIG. 2 is a cross-sectional view of the diaper depicting a unitized standing double cuff structure in accordance with an embodiment of the present invention;

FIG. 3 is a sectional view of the diaper showing details of the unitized standing double cuff structure;

FIG. 4 is a cross-sectional view of the diaper in accordance with an alternate embodiment of the present invention where the height of inner gather portion of the unitized standing double cuff structure is greater than that of outer gather portion;

FIG. 5 is a top plan view of a diaper in accordance with an embodiment of the present invention;

FIG. 6 is a partial cross sectional view of a diaper of the present invention depicting the unitized standing double cuff structure preventing body exudates from leaking out of the diaper.

### DETAILED DESCRIPTION OF THE INVENTION

With reference to the accompanying drawings, a novel design for a disposable absorbent article is described with the principles and embodiments as set out below. The novel design provides an improved mechanism for preventing the leakage of body exudates from a disposable absorbent article such as a diaper.

In accordance with the novel design, a disposable absorbent article is provided with a unitized standing double cuff structure. The double cuff structure is made of a center portion attached to the top sheet and two standing leg gathers, placed on both sides of the central portion. The double cuff structure can be manufactured using a single ribbon, with each edge of the ribbon forming a standing leg gather. The novel double cuff design is simpler to manufacture and is much more effective than existing designs in preventing the leakage of body exudates from the disposable absorbent article.

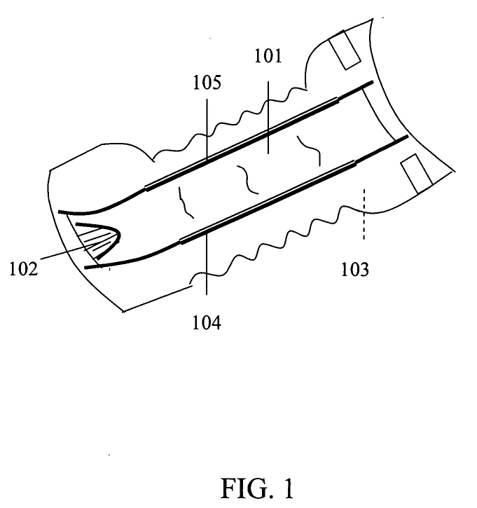

FIG. 1 shows a schematic view of a disposable article such as a diaper comprising a conventional unitized standing single cuff utilized for preventing leakage of body exudates from the diaper. It comprises top sheet 101, core 102, back sheet 103, and single cuffs 104 and 105. Top sheet 101 forms the upper surface of the diaper and contains apertures. Core 102 is fibrous and absorbs body exudates. Back sheet 103 forms the bottom surface of the diaper and is liquid impervious. Cuffs 104 and 105 are provided on the opposite sides of core 102 and are made of liquid impervious ribbons. A longitudinal elastic band is provided on the top ends of these cuffs.

The liquid released onto top sheet 101 of the diaper passes through the apertures and penetrates down to core 102 that absorbs it. The unabsorbed liquids flow laterally on top sheet 101 to the sides and reach cuffs 104 and 105. Cuffs 104 and 105 prevent the liquids from flowing out of the diaper.

However, in such a design, body exudates usually get accumulated near the cuffs. There is a possibility of the accumulated liquid leaking out from gaps formed between the cuffs and the body of the person wearing the diaper, for example when the wearer is in motion.

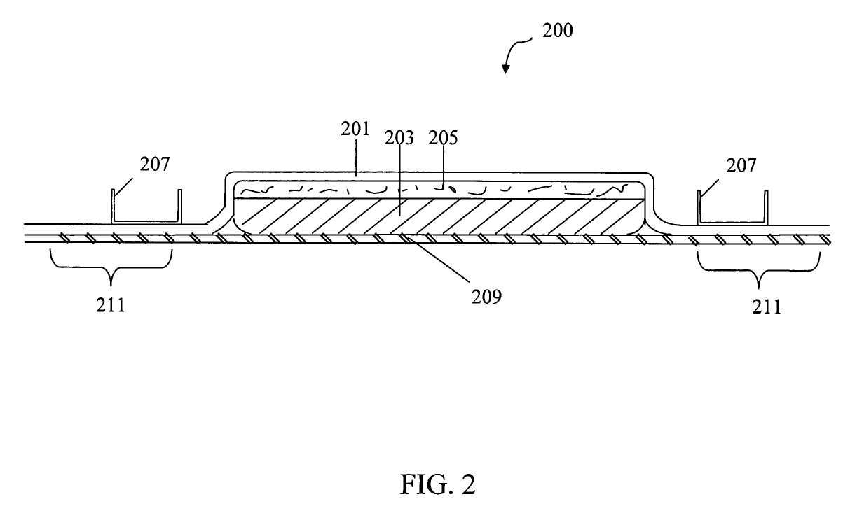

FIG. 2 shows a cross sectional view of a diaper 200 in accordance with an embodiment of the present invention. Diaper 200 comprises top sheet 201, absorbent core 203, isolating layer 205, unitized standing double cuff structures 207, back sheet 209 and a pair of laterally opposite base flaps 211. Base flaps 211 are formed from portions of top sheet 201 and back sheet 209, bonded together by an adhesive or welding. Body exudates pass through top sheet 201 to isolating layer 205 and finally to absorbent core 203. Isolating layer 205 is disposed between top sheet 201 and absorbent core 203, isolating both from each other and allowing body exudates to pass rapidly therethrough. Isolating layer 205 reduces the possibility that body exudates, which have been absorbed by the absorbent core 203, flow back to the top surface of absorbent core 203 and leak through top sheet 201. The body exudates are absorbed by absorbent core 203.

However, absorbent core 203 may not be able to retain all the body exudates and some exudates might leak back from absorbent core 203. These exudates flow laterally towards the sides of absorbent core 203. The exudates penetrate down from absorbent core 203 and move towards the edges of diaper 200. Unitized standing double cuffs 207 provided on both the edges of diaper 200 prevent the exudates from leaking out of diaper 200.

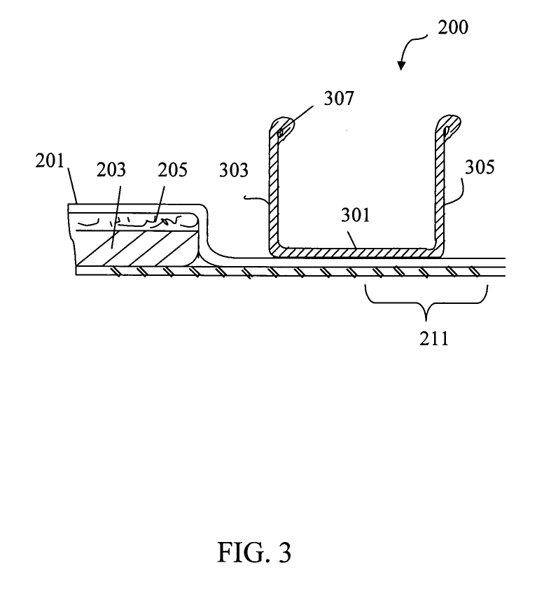

FIG. 3 is a sectional view of diaper 200 showing the details of unitized standing double cuff structure 207. Unitized standing double cuff structure 207 is made of a center portion 301 attached to top sheet 201 by adhesive or welding, and two standing leg gather portions 303 and 305, placed on both sides of central portion 301. Inner leg gather portion 303 is placed adjacent to absorbent core 203, while outer leg gather portion 305 is placed closer to the lateral edge of diaper 200. Inner leg gather portion 303 prevents exudates leaking from absorbent core 203 from moving out of diaper 200. In case some exudates manage to pass through inner leg gather portion 303, outer leg gather portion 305 prevents any further leakage from diaper 200.

Unitized standing double cuff structure 207 can be manufactured using a single ribbon, with each edge of the ribbon forming a standing leg gather. The ribbon can be made of liquid impervious material. Inner and outer leg gather portions 303 and 305 run along side the edge of the diaper and project above the upper surface of absorbent core 203. The center portion of the standing double cuff structure is typically from about 15 mm to about 21 mm, while the leg gather portions of the double cuff structure are typically in the range of from about 21 mm to about 36 mm. The individual leg gather portions can be of equal or unequal lengths. If the leg gather portions have equal lengths, the center portion is typically 21 mm, and the leg gathers are 28 mm. Alternatively, the center portion can be 15 mm, and the leg gathers can be 31 mm. Unequal leg gathers can typically have the following dimensions: 21 mm×20 mm×35 mm or 25 mm×15 mm×36 mm.

A longitudinal elastic band 307 is adhesively interposed at the two free edges of the single ribbon. Inner and outer leg gather portions 303 and 305 are maintained upright under a tensile force due to the contractibility of elastic band 307 contained therein. When the diaper is placed on the wearer, the unitized standing double cuff structure is caused to laterally expand, thus forming a desirable snug fitting area having extendible dimensions with elastic tension.

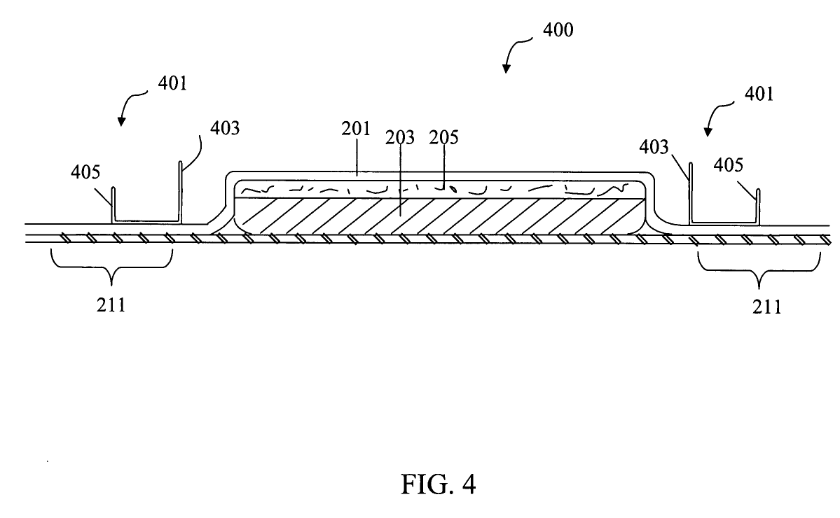

FIG. 4 is a cross-sectional view of a diaper 400 in accordance with another embodiment of the invention. As shown, unitized double cuff structure 401 is designed such that the height of inner leg gather portion 403 is greater than that of outer leg gather portion 405.

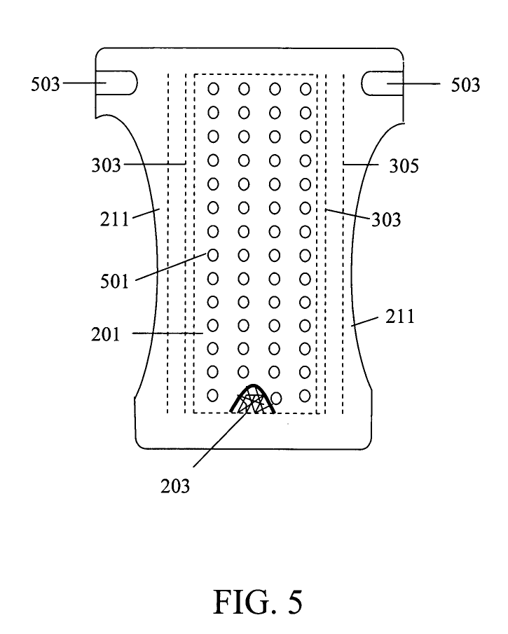

FIG. 5 is a top plan view of an embodiment of a disposable absorbent article, e.g., a diaper, constructed according to the present invention and being unfolded. FIG. 5 shows top sheet 201, absorbent core 203, base flaps 211, inner and outer standing leg gathers 303 and 305. Top sheet 201 is liquid permeable and forms the top surface of the diaper. Top sheet 201 is provided with multiple apertures 501 on its surface extending therethrough. Body exudates pass through these apertures onto absorbent core 203. As shown, inner and outer standing leg gathers 303 and 305 provided on two opposite sides of the absorbent core extend longitudinally throughout the length of the diaper. Inner and outer standing leg gathers 303 and 305 prevent the outflow of body exudates from the diaper. Tape fasteners 503 are provided to fasten the diaper about the body of the wearer.

Referring now to FIGS. 2 to 5, top sheet 201 is formed from a material such as a fibrous non-woven fabric or porous plastic film. Preferred non-woven materials include spun-bonded polypropylene, spun-bonded polyethylene, and thermally bonded webs of staple fibers. Back sheet 209 is made from a material such as a plastic film or laminate sheet of plastic film. The plastic film is preferably liquid-impermeable. Such material should be hydrophobic, soft in texture, and strong in tensile strength. An example includes hydro-entangled non-woven webs, which may contain some cotton and/or rayon fibers blended in with thermal-plastic fibers. Cellulose fibers can also be blended in small amounts to reduce costs. Other materials for forming back sheet 209 may include polypropylene films, co-extruded films (polyethylene and ethylene vinyl acetate), co-polymer films (polyethylene/polypropylene), and polylaminates (polypropylene nonwoven and polyethylene film).

Absorbent core 203 can be made from any suitable absorbent material, as well as combinations of different types of absorbent materials. For example, the absorbent core may be formed from a mixture of pulp fluff and superabsorbent particles wrapped in a liquid permeable tissue wrap. The superabsorbent particles may be substantially homogeneously mixed with hydrophilic fibers, or may be non-uniformly mixed. The pulp fluff may be exchanged with synthetic, polymeric, meltblown fibers or with a combination of meltblown fibers and natural fibers. Examples of superabsorbent materials include polyacrylamides, polyvinyl alcohol, polyacrylates, various grafted starches, and the like. A desired super absorbent material is a cross-linked polysodium acrylate, which can be purchased from BASF Corporation of Portsmouth, Va., under the trademark ASAP 2260. Absorbent core 203 may be bonded to the top sheet 201 and back sheet 209 with adhesive bonding or welding. Elastic bands 307 comprise material such as thread- or tape-like rubber, tape-like plastic foam or plastic film. The elastic bands can typically be formed from INVISTA lycra, spandex, FULFLEX rubber or similar materials.

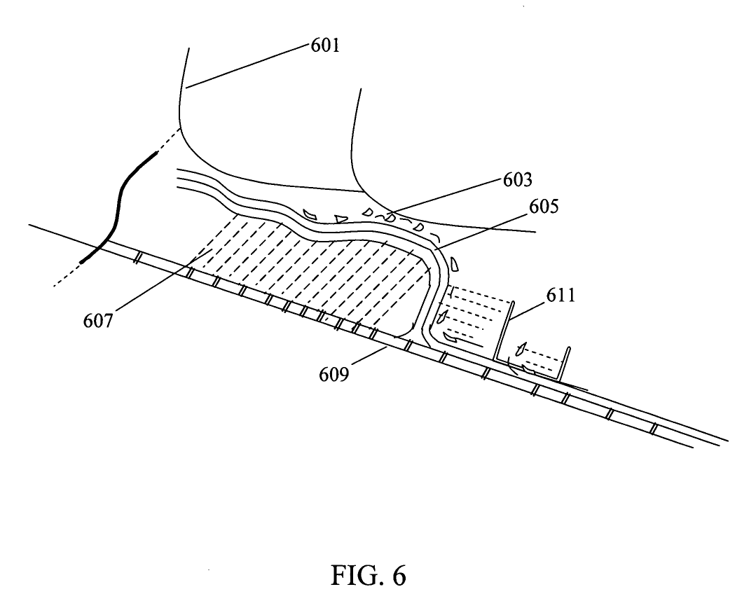

FIG. 6 is a partial cross sectional view of a diaper of the present invention depicting the double cuff structure preventing body exudates from leaking. FIG. 6 shows user 601, body exudates 603, top sheet 605, absorbent core 607, back sheet 609, and unitized standing double cuff structure 611. User 601 discharges exudates 603 onto top sheet 605, which pass through the apertures [not shown] into absorbent core 607. Absorbent core 607 absorbs a large quantity of body exudates 603. However, some exudates 603 remain on top sheet 605, or may seep back from absorbent core 607 to top sheet 605. Unitized standing double cuff structure 611, projecting above the surface of top sheet 605, prevents the exudates from reaching the edge of the diaper.

In the foregoing specification, specific embodiments of the present invention have been described. However, one of ordinary skill in the art will appreciate that various modifications and changes can be made without departing from the spirit or scope of the present invention as set forth in the claims below. Accordingly, the specification and figures are to be regarded in an illustrative rather than a restrictive sense, and all such modifications are intended to be included within the scope of present invention. The present invention can be used in other contexts, such as in other garments to retain liquids.

What is claimed is:

1. A disposable absorbent article comprising:
a liquid permeable top sheet;
a liquid impermeable back sheet;
an absorbent core interposed between the top sheet and the back sheet; and
a pair of unitized standing double cuff structures located outwardly with respect to the absorbent core along laterally opposite sides thereof, wherein the unitized standing double cuff structure is made of a single ribbon and wherein a center portion of the ribbon is attached to the topsheet and two edges of the ribbon form standing leg gathers placed on both sides of the center portion.

2. The disposable absorbent article of claim 1, wherein the unitized standing double cuff structure is liquid impermeable.

3. The disposable absorbent article of claim 1, wherein the unitized standing double cuff structure further comprises elastic bands affixed to the ribbon.

4. The disposable absorbent article of claim 1, wherein the absorbent article comprises an isolating layer.

5. The disposable absorbent article of claim 1, wherein the height of the standing leg gathers placed on both sides of the center portion of the ribbon are equal.

6. The disposable absorbent article of claim 1, wherein the height of the standing leg gather placed nearer to the absorbent core is greater than the height of the standing leg gather placed further away from the absorbent core.

7. The disposable absorbent article of claim 1, wherein the disposable absorbent article is a diaper.

8. The disposable absorbent article of claim 1, wherein the disposable absorbent article is a training pant.

9. A diaper comprising
a liquid permeable top sheet;
a liquid impermeable back sheet;
an absorbent core interposed between the top sheet and the back sheet;
an isolating layer disposed between the top sheet and the absorbent core;
a pair of base flaps located outwardly with respect to the absorbent core along laterally opposite sides thereof; and
a pair of unitized standing double cuff structures located inwardly with respect to the lateral edges of the base flaps and extending in a longitudinal direction along the diaper, wherein the unitized standing double cuff structure is made of a single ribbon and wherein a center portion of the ribbon is attached to the top sheet and two edges of the ribbon form standing leg gathers placed on both sides of the center portion.
